(19) United States
(12) Patent Application Publication
(10) Pub. No.: US 2009/0118772 A1
(43) Pub. Date: May 7, 2009

(54) POLYAXIAL BONE ANCHOR WITH INCREASED ANGULATION

(76) Inventors: Jennifer Diederich, Meriden, CT (US); Arnold Oyola, Northborough, MA (US); John Lipchitz, Chestnut Hill, MA (US)

Correspondence Address: CARR LLP (IST), 670 FOUNDERS SQUARE, 900 JACKSON STREET, DALLAS, TX, 75202, US

(21) Appl. No.: 12/131,725

(22) Filed: Jun. 2, 2008

Related U.S. Application Data
(60) Provisional application No. 60/941,584, filed on Jun. 1, 2007.

(51) Int. Cl. A61B 17/04

(52) U.S. Cl. 606/301; 606/305; 606/308

(57) ABSTRACT

Embodiments of a bone anchoring systems are disclosed. In one such embodiment, there is disclosed a bone anchoring system, comprising a bone anchor having a distal end portion adapted to engage a bone and a proximal end portion, a head coupled to the proximal end portion wherein the head is adapted to rotate with respect to the bone anchor, and a locking element coupled to the head and adapted to rotate with respect to the head to create additional rotation with respect to the bone anchor.

### CROSS REFERENCE TO RELATED APPLICATIONS

This application claims the benefit of the filing date of U.S. provisional patent application Ser. No. 60/941,584, entitled “Polyaxial Bone Anchors with Increased Angulation,” filed on Jun. 1, 2007, the disclosure of which is incorporated herein by reference for all purposes.

### TECHNICAL FIELD

The invention relates in general to skeletal stabilization delivery systems, and in particular to polyaxial bone anchors and methods for delivering and attaching polyaxial bone anchors to bony structures such as vertebrae.

### BACKGROUND INFORMATION

Polyaxial bone anchor systems are anchors in which one end is designed to be inserted into a boney structure and another end is allowed to either temporarily or permanently angularly pivot about multiple axes relative to the end of the bone anchor that is fixed to the boney structure. Typically, polyaxial bone anchors are attached to other medical implant components such as plates, rods, cables, or linking components. These polyaxial anchors may be used in dynamic or static fusion medical implant systems.

Polyaxial anchors are typically used to connect one element of a medical implant device at an angle relative to another component. In some medical implant systems, polyaxial bone anchors may be used capture one element of an implant relative to another and to then lock the two relative to one another to prevent subsequent movement therebetween. In some systems, the failure to properly lock two elements of a medical implant together may result in failure of the implant and possible serious injury to the patient within which the implant is placed.

For instance, orthopedic injuries, deformities, and degenerative diseases often require intervention in the form of surgery for placing implants to stabilize an internal structure, promote healing, and relieve pain. In the area of spinal surgery, for example, a common procedure includes placement of bone securing elements in the form of screws or hooks that are joined by a connecting rod spanning between these elements. Once placed, the rod must be firmly secured to the bone securing elements to provide a stable construct which effectively immobilizes a corresponding portion of the spine. Relatively large forces may be applied to the construct in the form of a set screw or locking element which presses firmly against the rod to secure the rod to the bone securing element (e.g., pedicle screw head or other anchor element).

As an example configuration of a vertebral stabilization implant device, a first pedicle screw may be implanted into a first vertebral body of a spine and a second pedicle screw may be implanted into another vertebral body at another vertebral level. A distraction rod or brace may then be used to connect the first and second pedicle screws. In some instances, the head of the pedicle screws (or a receiving member coupled thereto) is fork-shaped to receive a distracting rod or brace. With such heads, inner (female) threaded sections may be included within the head for receiving a set screw for applying direct or indirect pressure on a previously inserted distraction rod for securing such distraction rod relative to the pedicle screws.

In other systems, the head of the pedicle screws may be coupled to a pivot post, bearing post or locking screw which are designed to couple to a dynamic link. One such system is described in commonly assigned U.S. patent application Ser. No. 11/852,821, entitled “Offset Dynamic Motion Spinal Stabilization System,” filed on Sep. 10, 2007. The disclosure of which is incorporated herein by reference for all purposes.

Many existing polyaxial head anchors limit the pivotable movement or angulation between the longitudinal axis of the bone anchor and the polyaxial head. With limited angulation, it may be difficult for a surgeon to attach a rod or a dynamic link to the respective heads of the bone anchors. In some cases the connecting member, such as a plate, rod or other device must be bent to connect two bone anchors. The bending of connecting devices is not a preferred method because this may weaken the connecting device.

Additionally, it is generally desirable for spinal implants to maintain a small profile so as to minimize the impact upon the patient. Consequently, it is desirable to have low or small profile heads for the polyaxial anchors. However, in some instances, the loading required to lock the components can cause component deformation in these low profile implants, resulting in assembly loosening and possible implant failure.

What is needed, therefore, is a polyaxial bone anchor which allows for greater angular movement than existing polyaxial anchors. It is also desirable that such polyaxial bone anchors have small profile polyaxial heads which maintain the required structural strength.

### SUMMARY

In response to these and other problems, in one embodiment, there is disclosed a bone anchoring system, comprising a bone anchor having a distal end portion adapted to engage a bone and a proximal end portion, a head coupled to the proximal end portion wherein the head is adapted to rotate with respect to the bone anchor, and a locking element coupled to the head and adapted to rotate with respect to the head to create additional rotation with respect to the bone anchor.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

FIG. 1a is a side view of one embodiment of a polyaxial bone anchor system in a first or in-line position.

FIG. 1b is a side view of the embodiment of FIG. 1a in a second or angulated position.

FIG. 2 is an isometric exploded view of the embodiment of FIG. 1a.

FIG. 3 is a cross-section view of the embodiment of FIG. 1a.

FIG. 4a is a side view of one embodiment of a polyaxial bone anchor system in a first or in-line position.

FIG. 4b is a side view of the embodiment of FIG. 4a in a second or angulated position.

FIG. 5 is an exploded view of the embodiment of FIG. 4a.

FIG. 6 is a cross-section view of the embodiment of FIG. 4a.

FIG. 7a is a partial cross-section view of the embodiment of FIG. 4a showing angulation between the anchor and the head

FIG. 7b is a partial cross-section view of the embodiment of FIG. 4a. showing angulation between the head and the locking member.

FIG. 8a is a side view of one embodiment of a polyaxial bone anchor system in a first or in-line position.

FIG. 8b is a side view of the embodiment of FIG. 8a in a second or angulated position.

FIG. 9 is an exploded view of the embodiment of FIG. 8a.

FIG. 10a is a partial isometric view of certain components of the embodiment of FIG. 8a.

FIG. 10b is a partial sectional isometric view of certain components of the embodiment of FIG. 8a.

FIG. 11 a cross-section view of the embodiment of FIG. 8a.

FIG. 12a is a partial cross-section view of the embodiment of FIG. 8a showing angulation between the anchor and the head.

FIG. 12b is a partial cross-section view of the embodiment of FIG. 8a. showing angulation between the head and the locking member.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

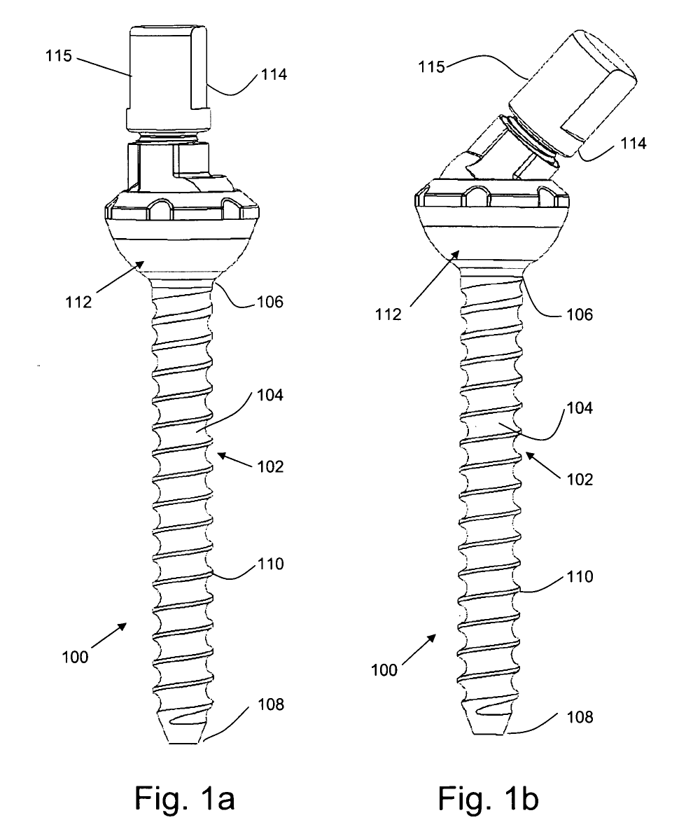

Turning now to FIG. 1a, there is presented one embodiment of a polyaxial bone anchor system 100 in a first or in-line position. FIG. 1b illustrates the polyaxial bone anchor system 100 in a second or angled position. In the illustrated embodiment, the polyaxial bone anchor system 100 may comprise an anchor 102 having a shank 104 which has a proximal end 106 and a distal end 108. For purposes of this application, the term “proximal” refers to the direction that points toward a user when the component is implanted in a patient or used by the user during a surgical procedure. Similarly, the term “distal” refers to the direction that points away from the user when the component is implanted in a patient or used by the user during a surgical procedure.

In this example embodiment, the anchor 102 is a screw and, thus has a helical thread 110 positioned about the shank 104. It is important to note that although a screw is illustrated, the anchor 102 could be any suitable anchor having any suitable surface. For example, the anchor 102 could be a ring shank fastener, a barb, a nail, a brad or a trocar. Furthermore, the anchor 102 may also have an expandable diameter which allows the anchor to “lock” into the bone after placement.

In some embodiments, the shank 104 may be longitudinally cannulated to couple to a guide wire (not shown). In other embodiments, there may be an off-set bore (not shown in FIGS. 1a and 1b) that extends from the center of a distal end 108 to the side of the shank 104. Such an off-set bore is more fully described in commonly assigned U.S. patent application Ser. No. 10/991,845, entitled “Offaxis Anchor Guidance System,” filed on Nov. 18, 2004. The disclosure of which is incorporated herein by reference for all purposes. As used in this application, the term “bore” refers to a generally cylindrical shaped opening or hole.

The proximal end 106 of the shank 104 may be coupled to an anchor head 112. As will be described in more detail below, in certain embodiments, the anchor head 112 may be rotatedly coupled to a locking member, such as a set screw 114 which is capable of rotating relative to the shank 104 from a first or in-line position illustrated in FIG. 1a to a second or angulated position illustrated in FIG. 1b. In this illustrative embodiment, the proximal end portion 115 of the set screw 114 may be a bearing post having a diameter which allows it to rotatably couple to a dynamic brace (not shown) as described more fully in the above mentioned U.S. patent application Ser. No. 11/852,821. In other embodiments, the proximal portion 115 of the set screw 114 may be a forked shaped element for receiving a rod or other spinal brace (not shown).

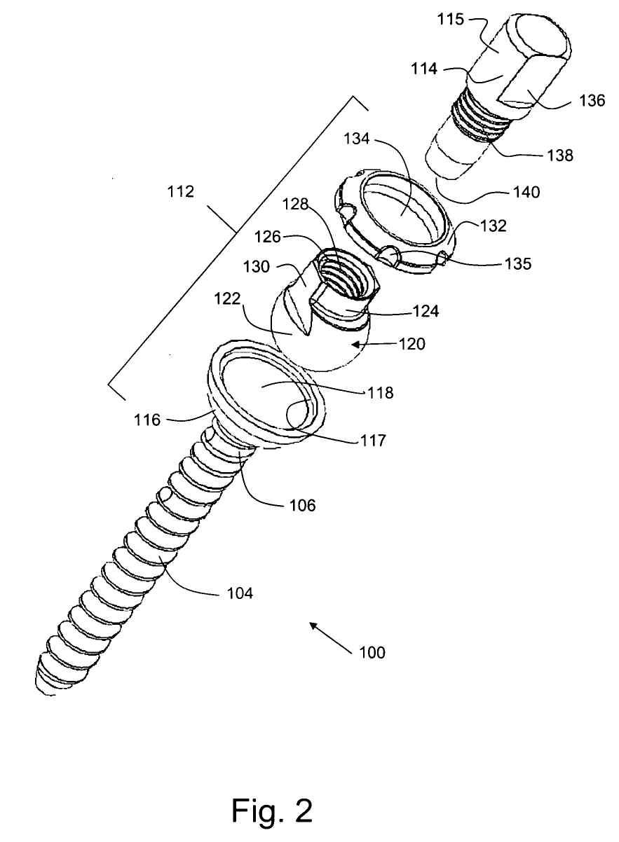

FIG. 2 is an exploded view of the polyaxial bone anchor system 100 and the anchor head 112. As illustrated, the proximal end 106 of the shank 104 is coupled to a partially spherical portion 116 of the anchor head 112. The proximal end of the spherical portion 116 opens to form a partially spherical socket 118. In certain embodiments, there is a plurality of helical interior threads 117 around an inside edge of the socket 118.

In certain embodiments, a coupling member 120 may be sized to partially fit within the socket 118. In some embodiments, the coupling member 120 may contain a ball or spherical portion 122 having a radius which is slightly smaller than the interior radius of the socket 118 so that the ball portion 122 can freely rotate within the socket 118. In some embodiments, a neck 124 projects out from one side of the ball portion 122. A bore 126 runs through the neck 124 and through the ball portion 122. The bore 126 is “offset” with respect to the ball portion 122. In other words, the longitudinal axis of the bore 126 and the center of the sphere of the ball portion 122 do not intersect. The offset nature of the bore 126 allows for greater angulation. In certain embodiments, there may be a plurality of internal helical threads 128 running along a portion of the bore 126. In some embodiments, there may be plurality flats 130 to allow coupling to a surgical instrument for adjusting the position of the coupling member 120 relative to the socket 118.

A ring or circular cap 132 couples to the spherical portion 116 of the anchor 104. A circular opening 134 is formed within the circular cap 132 which is sized to allow angular movement of the neck 124 of the coupling member 120 with respect to the anchor 104. In certain embodiments, a lip (not shown) projecting from a distal face (not shown) of the circular cap 134 includes a plurality of helical exterior threads sized to mate with the helical interior threads 117 of the spherical portion 116 of the anchor 104. Thus, the circular cap 132 can be threadingly mated to spherical socket 118 and thus securing the ball portion 122 of the coupling member 120 within the socket while allowing the neck 124 to rotate with respect to the anchor 104. In other embodiments, the circular cap 132 may be welded, press fit or permanently attached using techniques or combinations thereof known in the art. In certain embodiments, there may be a plurality of indentations 135 sized to couple to a torque transmitting instrument (not shown) so that the circular cap 132 may be secured to the spherical portion 116 of the anchor 104. In other embodiments, once the circular cap 134 has been permanently secured to the spherical portion 116, the indentations 135 may be used to couple to a torque transmitting instrument to drive the screw shank 204 into a bone.

In certain embodiments, the proximal portion 115 of the set screw 114 is designed to act as a pivot pin or bearing post for a dynamic brace (not shown). In certain embodiments, there may be a plurality of flats 136 which couple to a torque transmitting instrument to turn the set screw 114. In other embodiments, the proximal end of the set screw 114 may have a torx indentation or another torque transmitting indentation to coupling to a torque transmitting instrument to turn the set screw. In some embodiments, next to the proximal portion 115, there may be a plurality of exterior helical threads 138 sized to mate with the interior helical threads 128 defined within the bore 126 of the coupling member 120. An engaging surface 140 may be defined on the distal end of the set screw 114 for engaging the surface of the spherical socket 118 of the spherical portion of the head 116.

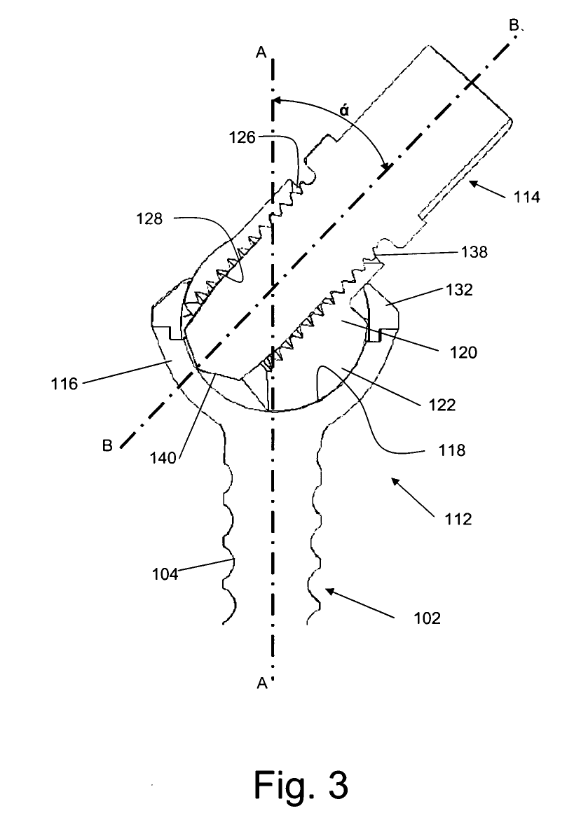

Turning now to FIG. 3, there is illustrated a detailed cross-sectional view of the assembled anchor head 112. As illustrated, the shank 104 of the anchor 102 expands to form the spherical portion 116. A portion of the ball portion 122 of the coupling member 120 is positioned within the spherical socket 118. In this example, the circular cap 132 is threadingly coupled to the spherical portion 116 and secures the ball portion 122 longitudinally while allowing the ball portion to rotate with respect to the shank 104. The exterior threads 138 of the set screw 114 engage the interior threads 128 of the bore 126 of the coupling member 120. As illustrated, the set screw 114 may be threadingly rotated with respect to bore 126 such that the engaging surface 140 may engage the surface of the socket 118 to secure and lock the position of the set screw 114 with respect to the shank 104. In certain embodiments, the engaging surface 140 may be cone shaped to concentrate stresses on the interior surface of the socket 118. In other embodiments the engaging surface 140 may be grooved, roughened, knurled, textured or otherwise modified to increase the friction between the engaging surface and the surface of the socket 118.

A longitudinal axis A-A of the shank 104 intersects a longitudinal axis B-B of the set screw 114 to form an angle \acute{α}. As can be seen in FIG. 3, the offset bore 126 provides for greater angulation than if the bore were aligned with the ball portion 122 of the coupling member 120. In some example embodiments, angle \acute{α} may be greater 40 degrees.

### DESCRIPTION OF A SECOND EMBODIMENT

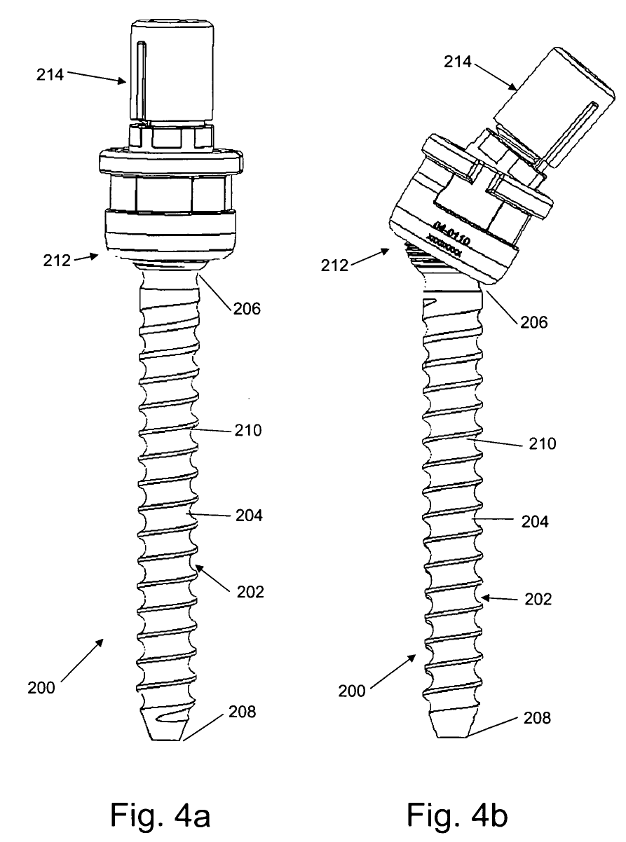

A second embodiment of a polyaxial bone anchor system is illustrated in FIGS. 4a and 4b. For brevity and clarity, some descriptions of those parts which are identical or similar to those described in connection with the above embodiment illustrated in FIGS. 1a through 3 will not be discussed in detail again. Reference should be made to the foregoing paragraphs with the following description to arrive at a complete understanding of this second embodiment.

Turning now to FIG. 4a, there is presented one embodiment of a polyaxial bone anchor system 200 in a first or in-line position. FIG. 4b illustrates the polyaxial bone anchor system 200 in a second or angled position. In the illustrated embodiment, the polyaxial bone anchor system 200 may comprise an anchor 202 having a shank 204 which has a proximal end 206 and a distal end 208. In this example embodiment, the anchor 202 is a screw and, thus has a helical thread 210 positioned about the shank 204. The anchor 202 in this embodiment may be similar to anchors described the applications referenced above or in commonly assigned U.S. patent application Ser. No. 10/989,782, entitled “Connector Transfer Tool for Internal Structural Stabilization Systems” filed on Nov. 16, 2004. The disclosure of which is incorporated herein by reference for all purposes.

The proximal end 206 of the shank 204 may be coupled to an anchor head 212. As will be described in more detail below, in certain embodiments, the anchor head 212 may be rotatedly coupled to the shank 204 and a locking member, such as a set screw 214 which is capable of rotating relative to the shank 204 from a first or in-line position illustrated in FIG. 4a to a second or angulated position illustrated in FIG. 4b.

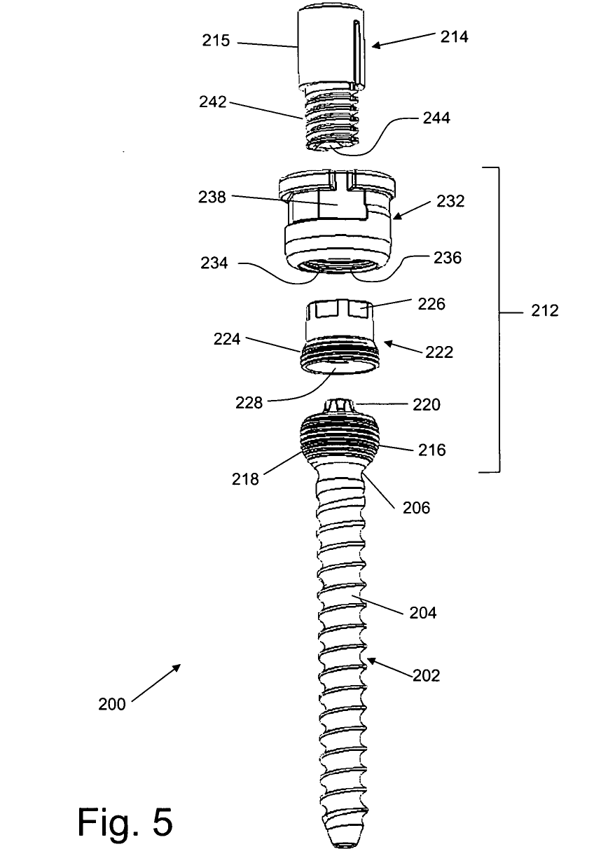

FIG. 5 is an exploded view of the polyaxial bone anchor system 200. As illustrated, the proximal end 206 of the shank 204 is coupled to a spherical portion or an head portion 216 of the anchor 202. In certain embodiments, the head portion 216 may be partially spherical in shape having a plurality of exterior threads 218 defined within the exterior surface of the spherical shape. In some embodiments, the exterior threads 218 may be reversed threaded so that the head portion 216 couples in a counter-clockwise rotation rather than the more traditional clockwise rotation. Formed on a proximal face of the head portion 216, there may be a torque engagement feature or a torque engagement projection 220 which is adapted to mate with a torque driving instrument (not shown) so that the bone anchor 202 can be screwed into a bone.

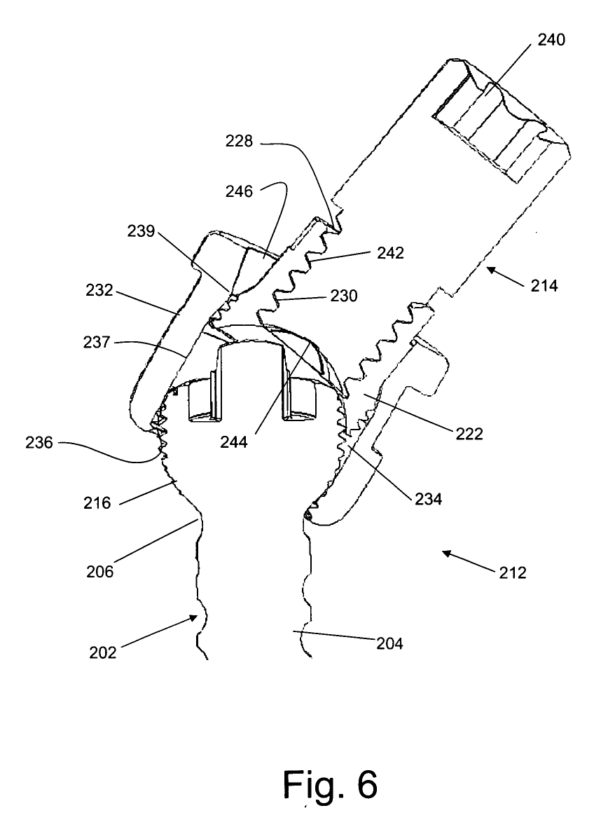

In certain embodiments, the anchor head 212 also includes a coupling member or collet 222. The collet 222 may be generally tubular in shape. In certain embodiments, the collet may have an enlarged distal portion having a series of external threads 224 defined along an exterior surface. On the proximal exterior surface portion, there may be a plurality of flats 226 which are sized to couple to a torque driver or other instrumentation (not shown). As will be explained in more detail with reference to FIG. 6, the collet 222 has a longitudinal bore 228 defined therethrough having a plurality of helical interior threads 230 (not shown in FIG. 5).

An anchor head housing 232 may be sized to house the collet 222 and a portion of the head portion 216 of the anchor 202. In certain embodiments, the anchor head housing 232 may be generally tubular in shape having a longitudinal bore 234 which may taper inward at its distal end. Defined within an interior surface of the longitudinal bore 234 may be a plurality of threads 236 adapted to engage the external threads 224 of the collet 222 and the external threads 218 on the head portion 216 of the anchor 204. Thus, in certain embodiments, during assembly, the collet 222 may be inserted into the anchor head housing 232 as the external threads 224 of the collet 222 engage the internal threads 236 of the anchor head housing until the external threads 224 are rotated past the internal threads 236. Similarly, the head portion 216 of the anchor 202 may be inserted into the anchor head housing 232 as the external threads 218 of the head portion 216 engage the internal threads 236 of the anchor head housing until the external threads 218 are rotated past the internal threads 236. Once assembled in this manner, the collet 222 and the head portion 216 are free to rotate independently of each other while being held within the anchor head housing 232.

In certain embodiments, there may be a plurality of flats 238 defined within the exterior surface of the anchor head housing 232 which are adapted to engage instrumentation (not shown) to allow positioning and stabilization of the anchor head housing 232.

In certain embodiments, the set screw 214 has a proximal portion 215 which may be design to act as a pivot pin or bearing post for a dynamic brace (not shown). In some embodiments, there may be a plurality of flats which couple to a torque transmitting instrument (not shown) to turn the set screw 214. In other embodiments, the proximal end of the set screw 214 may have a torx indentation 240 (FIG. 6) or another torque transmitting indentation for coupling to a torque transmitting instrument (not shown) to turn the set screw. In some embodiments, there may be a plurality of exterior helical threads 242 defined along a distal portion of the set screw 214 which are sized to mate with the interior helical threads 230 (FIG. 6) defined within the bore 228 of the collet 222. A curved engaging surface 244 may be defined at the distal end of the set screw 214 for engaging a surface of the head portion 216 of the anchor 202.

Turning now to FIG. 6, there is illustrated a detailed cross-sectional view of the assembled anchor head 212. As illustrated, the shank 204 of the anchor 202 expands at the distal end 206 to form the head portion 216 which has been threadedly inserted into the threaded distal end of the bore 234 of the anchor head housing 232. At a proximal portion 246 of the bore 234, the diameter of the bore 234 gradually increases which causes the bore expand at the proximal end. The expansion of the bore 234 allows for greater angulation of the set screw 214 with respect to the screw shank 204. As illustrated in FIG. 6, the diameter of the bore 234 varies as the bore progresses along its longitudinal axis. At the distal end, the bore has a first diameter which allows for the anchor head portion 216 to threadingly pass through. The diameter of the bore gradually increases to an intermediate point 237, which allows for an articulation of the anchor head portion within the bore 234. The diameter of the bore 234 then gradually narrows to a point 239. The narrowed diameter at point 239 restrains the collet 222, yet still allows some angulation of the collet. Finally, the diameter of the bore 234 increases as the bore approaches the proximal end of the anchor head housing.

Once the set screw 214 has been placed in its desired angular orientation, an instrument, such as a torx driver (not shown), may engage the torx indentation 240 to turn the set screw. The exterior threads 242 of the set screw 214 thus rotatingly engage the interior threads 230 defined within the longitudinal bore 228 of the collet 222. A clockwise rotation of the torx driver thus drives the set screw 214 in a distal direction until the curved engagement surface 244 engages the head portion 216. When this occurs, the collet 222 is forced up the screw and head portion 216 is forced down against the bore 236. Thus, once the curved engagement surface 244 is fully engaged with the head portion 216, the set screw 214, the collet 222, and head portion 216 are positionally secured and locked in place with respect to the shank 204.

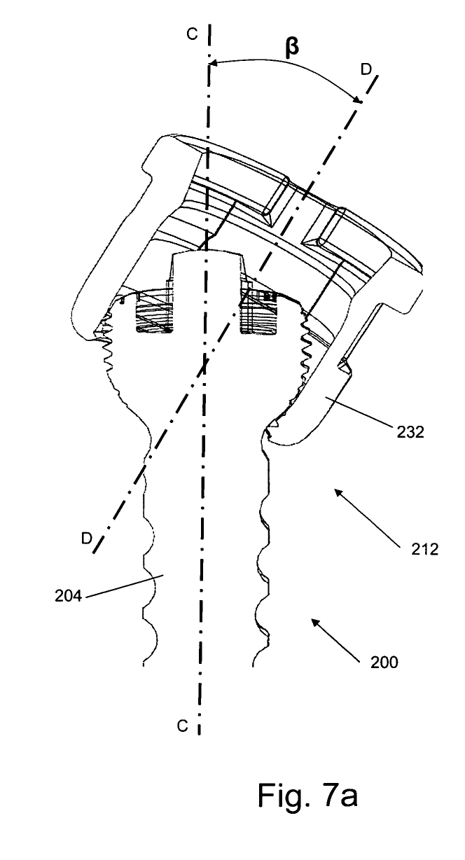

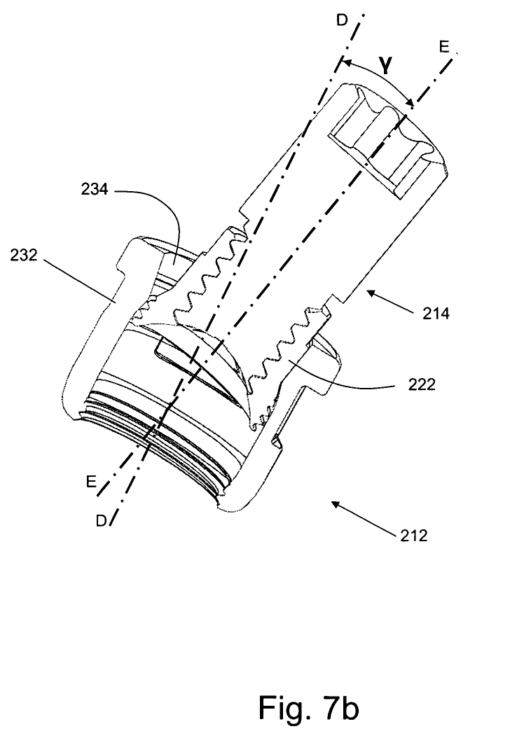

FIGS. 7a and 7b illustrate the additional angulation possible with the polyaxial bone anchor system 200. FIG. 7a is a cross section view of the screw head 212 with the set screw 214 and the collet 222 removed for clarity. The longitudinal axis of the anchor shank 204 may be represented by the line C-C. Similarly, the longitudinal axis of the anchor housing head 232 may be represented by the line D-D. As can be seen in FIG. 7a, with respect to the anchor shank 204, the anchor housing head 232 can achieve a maximum rotation at angle β which is the angle between the lines C-C and D-D.

FIG. 7b illustrates the anchor head 212 with the anchor head portion 216 and anchor shank 204 removed for clarity. The longitudinal axis of the set screw 214 may be represented by line E-E. The angle γ between the line D-D (the longitudinal axis of the anchor head housing 232) and the line E-E represents the additional angulation between the set screw 214 and the anchor head housing 232. This additional angulation is due to the use of combining the collet 222 the widening of the bore 234 at its proximal end. Thus, the total amount of angulation between the set screw 214 and the anchor 202 is the addition of the angle β and γ. In some example embodiments, it can be seen that the angle β may be approximately 30 degrees. The angle γ may be approximately 10 degrees. Therefore, the addition of β and γ may yield a total angulation of the set screw 214 with respect to anchor shank 204 of approximately 40 degrees or more.

### DESCRIPTION OF A THIRD EMBODIMENT

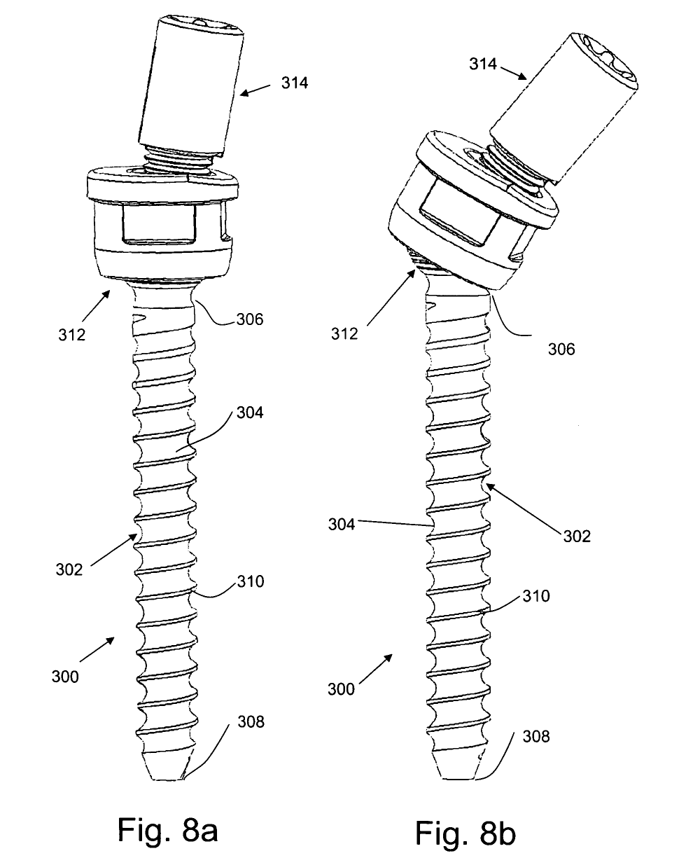

A third embodiment of a polyaxial bone anchor system is illustrated in FIGS. 8a and 8b. For brevity and clarity, some of the descriptions of those parts which are identical or similar to those described in connection with the above embodiment illustrated in FIGS. 1a through 7b will not be repeated here. Reference should be made to the foregoing paragraphs with the following description to arrive at a complete understanding of this second embodiment.

Turning now to FIG. 8a, there is presented one embodiment of a polyaxial bone anchor system 300 in a first or in-line position. FIG. 8b illustrates the polyaxial bone anchor system 300 in a second or angled position. In the illustrated embodiment, the polyaxial bone anchor system 300 may comprise an anchor 302 having a shank 304 which has a proximal end 306 and a distal end 308. In this example embodiment, the anchor 302 is a screw and, thus has a helical thread 310 positioned about the shank 304.

The proximal end 306 of the shank 304 may be coupled to an anchor head 312. As will be described in more detail below, in certain embodiments, the anchor head 312 may be rotatedly coupled to the shank 304 and a locking member, such as a set screw 314 which is capable of rotating relative to the shank 304 from a first or in-line position illustrated in FIG. 8a to a second or angulated position illustrated in FIG. 8b.

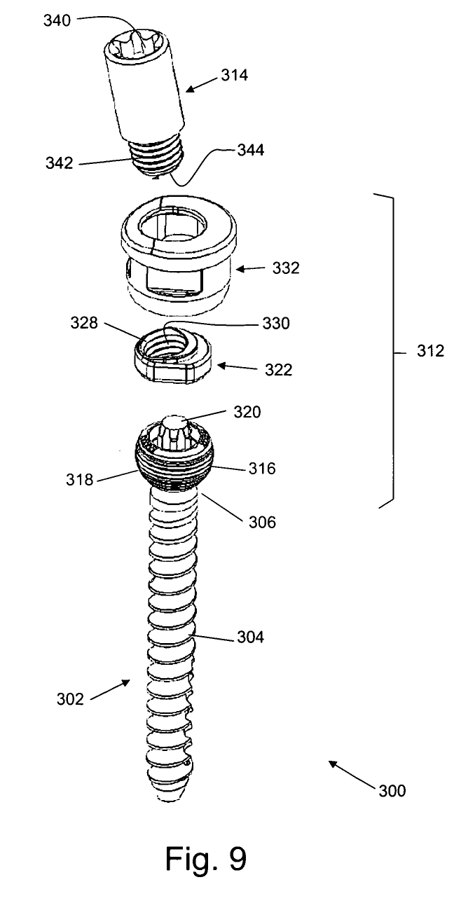

FIG. 9 is an exploded view of the polyaxial bone anchor system 300. As illustrated, the proximal end 306 of the shank 304 is coupled to a spherical or head portion 316 of the anchor 302. In certain embodiments, the head portion 316 may have a plurality of exterior threads 318 defined within a distal portion of the spherical shape. Formed on a proximal face of the head portion 316, there may be a torque engagement feature or torque engagement projection 320 which is adapted to mate with a torque driving instrument (not shown) so that the bone anchor 302 can be screwed into a bone.

In certain embodiments, the anchor head 312 includes an offset coupling member 322. In certain embodiments, the offset coupling member 322 may be generally puck like in shape. As will be explained in more detail below, the offset coupling member 322 has an offset bore 328 defined laterally therethrough having a plurality of helical interior threads 330.

An anchor head housing 332 may be sized to house the offset coupling member 322 and a portion of the head portion 316 of the anchor 302. In certain embodiments, the anchor head housing 332 may be generally tubular in shape.

In some embodiments, the proximal end of the set screw 314 may have a torx indentation 340 or another torque transmitting indentation for coupling to a torque transmitting instrument (not shown) to turn the set screw. In some embodiments, there may be a plurality of exterior helical threads 342 defined along a distal portion of the set screw 314 which are sized to mate with the interior helical threads 330 defined within the bore 328 of the offset coupling member 322. An engaging surface 344 may be defined at the distal end of the set screw 314 for engaging a surface of the head portion 316 of the anchor 302.

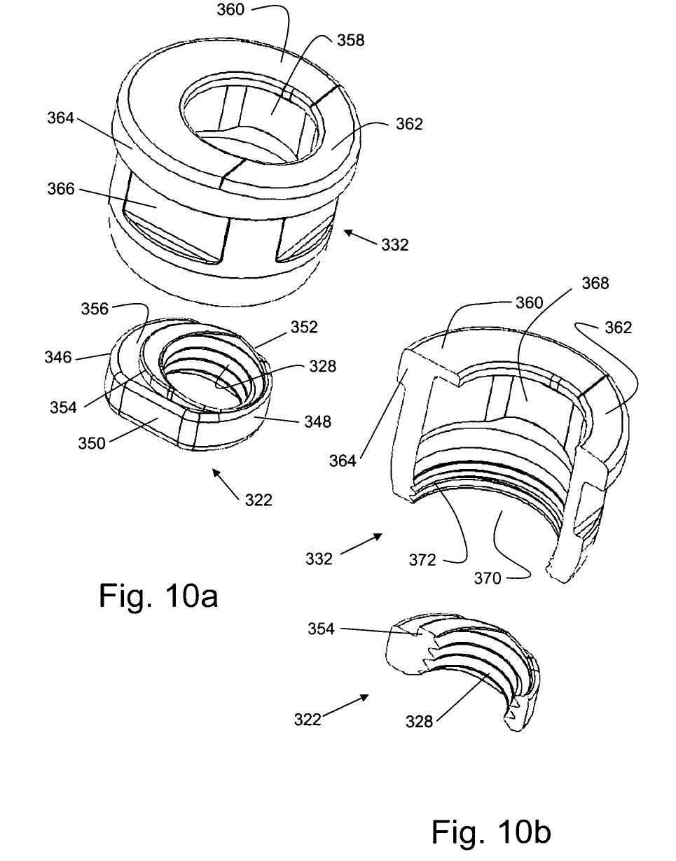

FIG. 10a is a detailed isometric view of the anchor head housing 332 and the offset coupling member 322. FIG. 10b is a partial isometric sectional view of the anchor head housing 332 and the offset coupling member 322. Referring now to both FIGS. 10a and 10b, the details of the offset coupling member 322 will be discussed. As described above, the offset coupling member 322 is an elongated disc shape with curve ends 346 and 348 and flat sides 350 and 352 (not shown). In certain embodiments, a small neck 354 projects away from a proximal face 356. The threaded bore 328 runs through the offset coupling member 322 at an offset angle to an axis that is perpendicular to the proximal face 356. In certain embodiments, the threaded bore is laterally positioned so the bore is laterally offset from the longitudinal axis of the anchor head housing when the coupling member 322 is assembled within the anchor housing member.

The neck 354 is sized to fit within an opening 358 defined in the proximal side of the anchor head housing 332. As illustrated in FIG. 10a, the proximal side of the anchor head housing 332 comprises two surfaces 360 and 362. Surface 360 is normal to a longitudinal axis of the anchor head housing 332. Surface 362 is sloped relative to surface 360 and from its intersection with the surface 360, surface 362 extends towards the distal end of the anchor head housing 332. Thus, a lip 364 that circles the proximal end of the anchor head housing 332 varies in thickness. In certain embodiments, there may be a plurality of flats 366 defined on the exterior surface of the head housing 332 which are adapted to engage instrumentation (not shown) to allow positioning and stabilization of the anchor head housing 332.

As can be seen from FIG. 10b, the anchor head housing 332 may be formed to house the offset coupling member 322 in a proximal portion of an interior cavity 368. The offset coupling member 322 may be sized such that it passes easily through a distal opening 370 of the head housing 332, but fits or “snaps” in place within a space within the interior cavity that keeps the offset coupling member from rotating. In other embodiments the offset coupling member 322 could be integral with the anchor head housing. The distal portion of the interior cavity 368 may be generally cylindrical in shape to accommodate the anchor head portion 316. In certain embodiments, there may be an inward tapering of the wall such that a diameter of a distal opening 370 is slightly smaller than the diameter of the distal portion of the interior cavity. Defined next to the distal opening 370 may be a plurality of interior threads 372 adapted to engage the external threads 318 on the head portion 316 of the anchor 304 (not shown). Thus, in certain embodiments, during assembly, the head portion 316 of the anchor 302 may be inserted into the anchor head housing 332 as the external threads 318 engage the internal threads 372 of the anchor head housing until the external threads 318 are rotated past the internal threads 372. Once assembled in this manner, the head portion 316 of the anchor 302 is held within the anchor head housing 332.

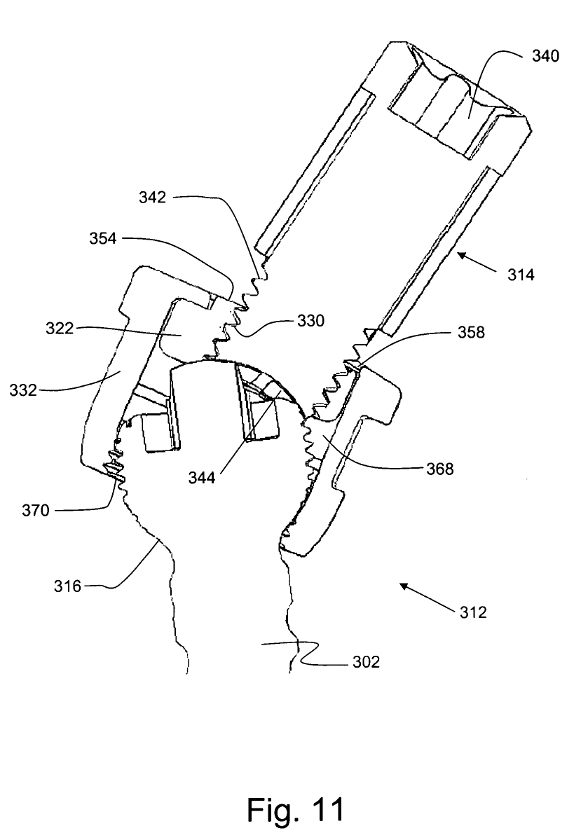

Turning now to FIG. 11, there is illustrated a detailed cross-sectional view of the assembled anchor head 312. As illustrated, the offset coupling member 322 has been inserted into the proximal portion of the interior cavity 368 with its neck 354 fitting within the opening 358. The head portion 316 has been threadedly inserted into the distal opening 370 of the anchor head housing 332 and maintains the offset coupling member 322 in the proximal portion of the interior cavity 368. The exterior threads 342 of the set screw 314 couple with the threads 330 of the bore of the offset coupling member 322. Once the set screw 314 has been placed in its desired angular orientation, an instrument, such as a torx driver (not shown), may engage the torx indentation 340 to turn the set screw. A clockwise rotation of the torx driver thus drives the set screw 314 in a distal direction until the engagement surface 344 of the set screw engages the head portion 316 of the anchor 302. Once the engagement surface 344 is fully engaged with the head portion 316, the set screw 314 is then positionally secured and locked in place with respect to the shank 304 of the anchor 302.

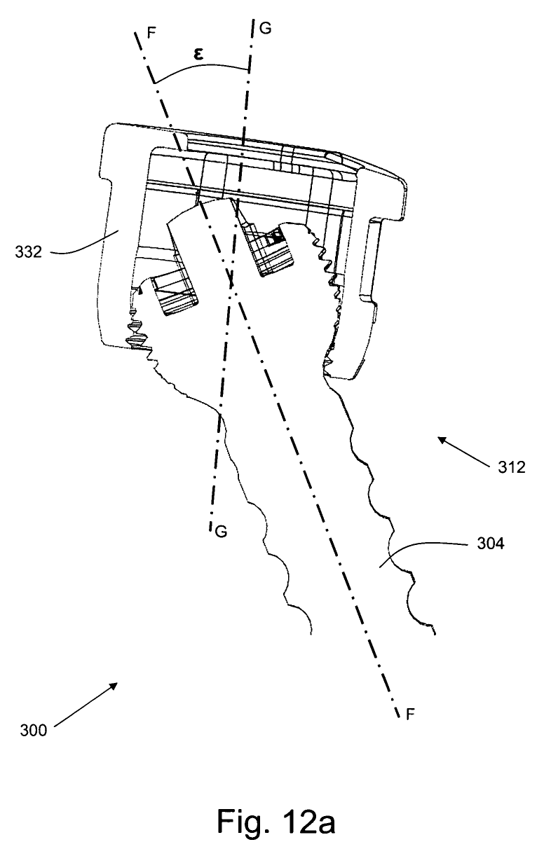

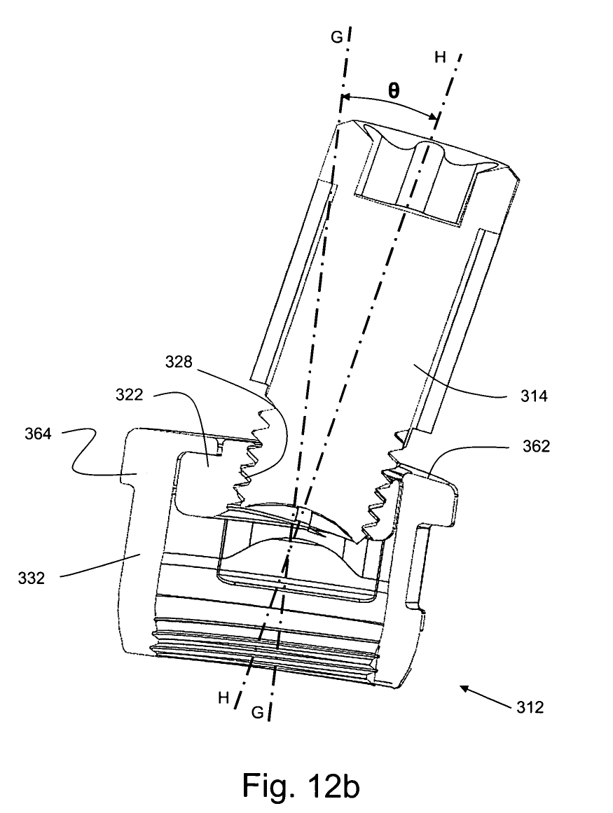

FIGS. 12a and 12b illustrate the additional angulation possible with the polyaxial bone anchor system 300. FIG. 12a is a cross section view of the screw head 312 with the set screw 314 and the offset coupling member 322 removed for clarity. The longitudinal axis of the screw shank 304 may be represented by the line F-F. Similarly, the longitudinal axis of the anchor housing head 332 may be represented by the line G-G. As can be seen in FIG. 12a, with respect to the screw shank 304, the anchor housing head 332 can achieve a maximum rotation at angle ε which is the angle between the lines F-F and G-G.

FIG. 12b illustrates the anchor head 312 with the head portion 316 removed for clarity. The longitudinal axis of the set screw 314 may be represented by line H-H. The angle θ between the line H-H and the line G-G represents the additional angulation between the set screw 314 and the anchor head housing 332. This additional angulation is due to the use of the offset bore 328 of the offset coupling member 322, including the lateral offset position of the center of the bore 328 with respect to the longitudinal axis G-G of the anchor head housing and the angular offset of the axis H-H of the bore with respect to the longitudinal axis G-G of the anchor head housing. Thus, the total amount of angulation is the addition of the angle ε and θ (FIG. 12a). Furthermore, the sloped surface 362 allows the set screw 314 to be fully engaged in extreme situations without interference from the lip 364 to assist in achieving greater angulation. This additional angulation may not be possible with conventional pedicle screw systems. Thus, the total amount of angulation between the set screw 314 and the anchor 302 is the addition of the angle ε and θ. In some example embodiments, it can be seen that the angle ε may be approximately 30 degrees. The angle θ may be approximately 10 degrees. Therefore, the addition of ε and θ may yield a total angulation of the set screw 314 with respect to anchor shank 304 of approximately 40 degrees or more.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

The foregoing description of the embodiments of the invention has been presented for the purposes of illustration and description. It is not intended to be exhaustive or to limit the invention to the precise form disclosed. Many combinations, modifications and variations are possible in light of the above teaching. Undescribed embodiments which have interchanged components are still within the scope of the present invention. It is intended that the scope of the invention be limited not by this detailed description, but rather by the claims appended hereto.

For instance, in some embodiments, there may be a bone fixation assembly comprising: a first coupling element having a inner surface defining a first generally spherical bore coaxial with a first longitudinal axis and a second generally spherical bore coaxial with a second longitudinal axis, wherein the first and second bores are in communication with one another; a bone anchor having a third longitudinal axis, a generally spherical head portion positioned within the first generally spherical bore and a threaded shank portion for insertion into bone; a second coupling element having a first generally spherical end portion positioned within the second generally spherical bore and an inner surface defining a bore coaxial with a fourth longitudinal axis; and a locking member having a distal end portion positioned within the bore of the second coupling element and rigidly coupled to the head portion of the bone anchor.

The above bone fixation assembly may include a configuration wherein the first, third and fourth longitudinal axis are not coaxial with one another.

In other embodiments, there may be a bone fixation assembly comprising: a bone anchor having a longitudinal axis, a distal threaded section and a semi circular head portion with a generally spherical inner surface; a coupling element having a center axis, a proximal end portion, a generally spherical distal end portion positioned within the generally spherical inner surface of the head portion and an inner surface defining a first bore that extends between the proximal end portion and the distal end portion, wherein the first bore is coaxial with a second longitudinal axis that is off set from the center axis; a locking ring rigidly fixed to the bone anchor and having an inner surface defining a second bore dimensioned to receive proximal end portion of the coupling element; and a locking member having a point compressed against the generally spherical inner surface of the head portion, wherein the locking member is threadingly coupled to first bore of the coupling element.

The above bone fixation assembly may include a configuration wherein the second longitudinal axis is offset and non coaxial with the longitudinal axis of the bone anchor.

The above bone fixation assembly may include a configuration wherein the locking ring has a plurality of recesses for transferring torque to the bone anchor.

The above bone fixation assembly may include a configuration wherein the locking member has at least one flat side surface for transferring torque.

In yet other embodiments, there may be a bone fixation assembly comprising: a first coupling element having a inner surface defining a first generally spherical bore coaxial with a first longitudinal axis and a second bore coaxial with a second longitudinal axis, wherein the first and second bores are in communication with one another and the first and second longitudinal axis are offset from one another; a second coupling element positioned within the second bore of the first coupling element, the second coupling element having an inner surface defining an angled bore with a third longitudinal axis that intersects the first longitudinal axis; a bone anchor having a fourth longitudinal axis, a generally spherical head portion positioned within the first generally spherical bore and a threaded shank portion for insertion into bone; and a locking member having a distal end portion positioned within the angled bore of the second coupling element and rigidly coupled to the head portion of the bone anchor.

1. A bone anchor system, comprising:
a bone anchor having a distal end portion adapted to engage a bone and a proximal end portion,
a partially spherical socket rigidly coupled to the proximal end portion of the bone anchor,
a coupling member including:
a partially spherical portion sized to rotatably engage an interior surface of the partially spherical socket,
an bore extending through the partially spherical portion of the coupling member wherein a longitudinal axis of the bore does not intersect a center of the partially spherical portion,
a ring coupled to an edge of the partially spherical socket for longitudinally securing the coupling member to the partially spherical socket,
a locking member longitudinally adjustably coupled to the bore, the locking element including:
a proximal portion adapted to engage a brace, and
a distal engagement portion having an engagement surface for engaging the interior surface of the partially spherical socket.

2. The bone anchor system of claim 1, wherein the locking member is a screw having a plurality of exterior threads positioned longitudinally between the proximal portion and the distal engagement portion, wherein the plurality of exterior threads are size to engage a plurality of interior threads defined within the bore.

3. The bone anchor system of claim 1, wherein the proximal portion of the locking member is generally cylindrical in shape.

4. The bone anchor system of claim 1, wherein the engagement surface of the locking member forms a cone.

5. The bone anchor system of claim 1, wherein the proximal portion of the locking member has a surface feature for coupling to a torque driving instrument.

6. The bone anchor system of claim 1, wherein the coupling member includes a proximal neck portion projecting from the partially spherical portion and extending around the bore, such that the neck portion extends through the ring when the partially spherical portion engages the interior surface of the partially spherical socket.

7. The bone anchor system of claim 1, wherein the ring comprises a plurality of indentions for coupling to a torque driving instrument.

8. A bone anchor system, comprising:
a bone anchor having a distal end portion adapted to engage a bone, a proximal end portion and a first longitudinal axis, wherein the proximal end portion includes:
a partially spherical portion,
a torque engagement feature positioned on a proximal side of the proximal end portion,
a anchor head housing having a longitudinal bore having a distal portion having a first diameter, a first intermediate portion having a second diameter, a second intermediate portion having a third diameter, and a proximal portion having a fourth diameter, wherein the first diameter and the third diameter are smaller than the second diameter,
a coupling member including:
a coupling bore extending longitudinally through the coupling member,
an enlarged distal end portion sized to rotatably fit within the first intermediate portion of the housing longitudinal bore and sized to have a exterior diameter which is greater than the third diameter of the second intermediate portion of the housing bore,
a locking member longitudinally adjustably coupled to the coupling bore, the locking member including:
a proximal portion adapted to engage a brace, and
a distal engagement portion having an engagement surface for engaging the proximal end portion of the bone anchor.

9. The bone anchor system of claim 8, wherein the first intermediate portion of the longitudinal bore of the anchor head housing is positioned between the distal portion and the proximal portion and the second intermediate portion is positioned between the first intermediate portion and the proximal portion.

10. The bone anchor system of claim 8, wherein the diameters of the longitudinal bore of the anchor head housing between the portions gradually changes from one longitudinal portion to an adjacent longitudinal portion.

11. The bone anchor system of claim 8, further comprising:
a plurality of internal threads defined within a surface of the distal portion of the longitudinal bore of the anchor head housing,
a plurality of external threads defined on an external surface of the enlarged distal end portion of the coupling member, wherein the plurality of external threads are sized to mate with the plurality of internal threads such that the enlarged distal end portion of the coupling member can be threadably inserted through the distal portion of the longitudinal bore of the anchor housing.

12. The bone anchor system of claim 8, further comprising:
a plurality of internal threads defined within the distal portion of the longitudinal bore of the anchor head housing,
a plurality of external threads defined on an external surface of the spherical portion of the bone anchor, wherein the plurality of external threads are sized to mate with the internal threads such that the spherical portion of the bone anchor can be threadably inserted through the distal portion of the longitudinal bore of the anchor housing.

13. The bone anchor system of claim 8, wherein the locking member is a screw having a plurality of exterior threads positioned longitudinally between the proximal portion and the distal engagement portion, wherein the plurality of exterior threads are size to engage a plurality of interior threads defined within the coupling bore.

14. The bone anchor system of claim 8, wherein the fourth diameter of the longitudinal bore of the anchor head housing is larger than the third diameter of the longitudinal bore providing for greater angulation between the anchor head housing and the coupling member.

15. A bone anchor system, comprising:
an anchor head housing having:
a first longitudinal axis,
an interior cavity extending about the first longitudinal axis, the interior cavity having a proximal interior portion and a distal interior portion,
a distal opening defined on a distal end of the housing extending to the distal cavity portion of the interior cavity,
a proximal opening defined on a proximal end of the housing running extending to the proximal cavity portion of the interior cavity,
a coupling member positioned at a predetermined fixed orientation within the proximal interior portion of the interior cavity, the coupling member having a coupling bore extending through the coupling element such that when the coupling member is positioned at the predetermined fixed orientation, the longitudinal axis of the coupling bore is not parallel to the longitudinal axis of the housing,
a bone anchor having a distal end portion adapted to engage a bone and a proximal end portion herein the proximal end portion includes:
a partially spherical portion partially positioned within the distal cavity portion of the anchor head housing,
a torque engagement feature positioned on a proximal side of the proximal end portion,
a locking member longitudinally adjustably coupled to the coupling bore, the locking member including:
a proximal portion adapted to engage a brace, and
a distal engagement portion having an engagement surface for engaging a surface of the proximal end portion of the bone anchor.

16. The bone anchor system of claim 15 further comprising:
a plurality of internal threads defined within the distal portion of the longitudinal bore of the housing,
a plurality of external threads defined on an external surface of the spherical portion of the bone anchor, wherein the plurality of external threads are sized to mate with the internal threads such that the spherical portion of the bone anchor can be threadably inserted through the distal portion of the longitudinal bore of the anchor housing.

17. The bone anchor system of claim 15, wherein the locking member is a screw having a plurality of exterior threads positioned longitudinally between the proximal portion and the distal engagement portion, wherein the plurality of exterior threads are size to engage a plurality of interior threads defined within the bore.

18. The bone anchor system of claim 15, further comprising:
a first surface defined on the proximal side of the anchor head housing wherein the surface is substantially normal to the first longitudinal axis, and,
a second surface defined on the proximal side of the anchor head housing adjacent to the first surface, wherein the second surface forms an obtuse angle with the first surface.

19. The bone anchor system of claim 15, wherein the longitudinal axis of the coupling bore is laterally offset from the longitudinal axis of the anchor head housing.

20. The bone anchor system of claim 15, wherein the proximal opening is not coaxial with the longitudinal axis of the anchor head housing.
